(19) United States
(12) Patent Application Publication
(10) Pub. No.: US 2015/0359654 A1
(43) Pub. Date: Dec. 17, 2015

(54) EXTERNAL NASAL DILATOR AND METHOD OF USE

(71) Applicants: LEONARD VINCENT BENTIVEGNA, YORK, PA (US); LEONARD CHARLES BENTIVEGNA, YORK, PA (US)

(72) Inventors: LEONARD VINCENT BENTIVEGNA, YORK, PA (US); LEONARD CHARLES BENTIVEGNA, YORK, PA (US)

(21) Appl. No.: 14/711,830

(22) Filed: May 14, 2015

Related U.S. Application Data
(60) Provisional application No. 62/012,040, filed on Jun. 13, 2014.

(51) Int. Cl. A61F 5/08

(52) U.S. Cl. CPC A61F 5/08

(57) ABSTRACT

An external nasal dilator formed as a single body truss member, the truss member comprising (1) an upper and lower surface; (2) a first end region with an engagement means to engage the alar facial groove and a second end region with an engagement means to engage the area anterior to the tragus of the ear; and (3) an intermediate segment connecting the first and second regions and configured to traverse the cheek located between the alar facial groove and area anterior to the tragus of the ear. The external dilators may be used in a pair to form a system for dilating nasal passages on opposite sides of the face.

### RELATED APPLICATIONS

This application claims priority to U.S. Provisional Application No. 62/012,040 filed Jun. 13, 2104. The entire contents of the above application are hereby incorporated by reference as though fully set forth herein.

### FIELD

The present invention relates to the field of nasal dilators. More specifically, the present invention relates to external nasal dilators applied to the face.

### BACKGROUND

Millions of people suffer everyday from common breathing issues from snoring, to allergies, to colds and flus that inhibit their airflow and adversely impact their lives. It is typical to experience partial or complete nasal congestion, blockage, and/or obstruction/nasal constriction at some point in their lifetime. The congestion can be caused by conditions such as allergic reactions, common cold, sinusitis, deviated septum, nasal polyps and reaction to medication. While the effects of nasal constriction can be merely a mild annoyance such as snoring, headaches and/or facial pain, it can also cause life-threatening conditions such as sleep apnea, hypoxia, right-sided heart failure, and other respiratory distress.

Nasal dilators are often used to relieve this congestion and they work by opening the nostril or nasal passage to improve airflow through the nose. Nasal dilators may be broadly classified as external or insertable. For external nasal dilators, there has only been a few approaches disclosed in the prior art, namely to place a strip across the bridge of the nose applied in several different variations or to place a strip across the cheekbone. Most existing external nasal dilators are placed across the bridge of the nose and rely on a thin polymer spring contained in the nasal strip to lift open nasal passages from the top of the nostrils. The existing nasal strips on the market are only able to open a smaller portion of the nasal airways. Therefore, it would be an improvement to provide a nasal dilator that covers a larger surface area and opens the nasal flares to provide an effective alternative to external nasal dilator strips known in the prior art.

### BRIEF SUMMARY OF THE INVENTION

It is the object of the present invention to address several challenges in previous attempts to design external nasal dilators by providing an external nasal dilator that covers a larger surface area while effectively opening the nasal flares. The present invention is an external nasal dilator formed as a single body truss member, the truss member comprising (1) an upper and lower surface; (2) a first end region with an engagement means to engage the alar facial groove and a second end region with an engagement means to engage the area anterior to the tragus of the ear; and (3) an intermediate segment connecting the first and second regions and configured to traverse the cheek located between the alar facial groove and area anterior to the tragus of the ear. The external dilators may be used in a pair to form a system for dilating nasal passages on opposite sides of the face.

In another embodiment of the present invention, a method of dilating nasal passages is disclosed, the method comprising providing a first truss member having (a) a first end region with an engagement means to engage the alar facial groove, (b) a second end region with an engagement means to engage the area anterior to the tragus of the ear and (c) an intermediate segment connecting the first and second regions wherein the intermediate segment is configured to traverse the check located between the alar facial groove and area anterior to the tragus of the ear. The truss member includes a flexible strip of base material with an upper and lower surface defining the first and second end regions and the intermediate segment and wherein the engagement means is an adhesive material along the lower surface of the flexible base material. The adhesive material is placed at the first end region along the alar facial groove and along the area anterior to the tragus of the ear.

### BRIEF DESCRIPTION OF THE DRAWINGS

FIG. 1A. FIG. 1A is front view of the truss member and its upper surface.

FIG. 1B. FIG. 1B is back view of the truss member and its lower surface.

FIG. 2. FIG. 2 is a front view showing placement of the truss member on a user's face.

FIG. 3. FIG. 3 is an exploded perspective view of the three layers of the truss member.

FIG. 4. FIG. 4 is a front view showing placement of the system of the present invention on a user's face.

### DETAILED DESCRIPTION

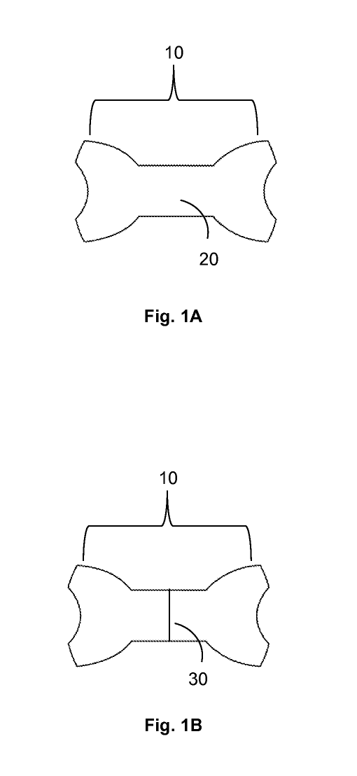

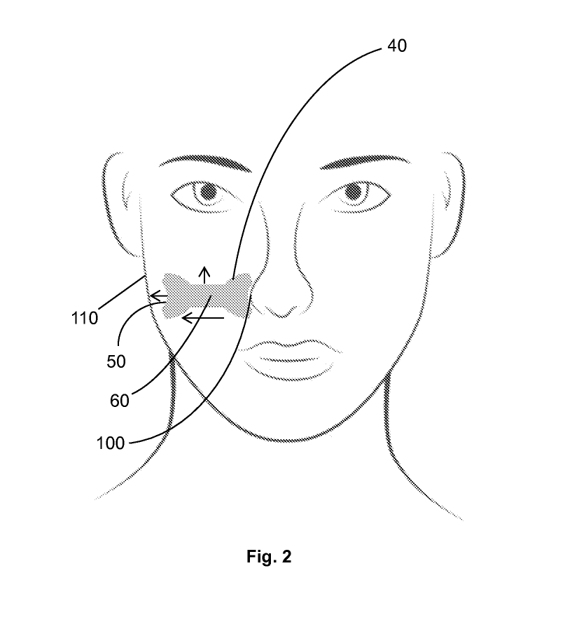

Turning to FIGS. 1A and B, an external nasal dilator is formed as a single body truss member 10. The truss member has an upper surface 20 and lower surface 30. As shown in FIG. 2, the first end region 40 has an engagement means to engage the alar facial groove 100 and a second end region 50 with an engagement means to engage the area anterior to the tragus of the ear 110. An intermediate segment 60 connects the first end region 40 and second end regions 50 and is configured to traverse the cheek 120 located between the alar facial groove 100 and area anterior to the tragus of the ear 110.

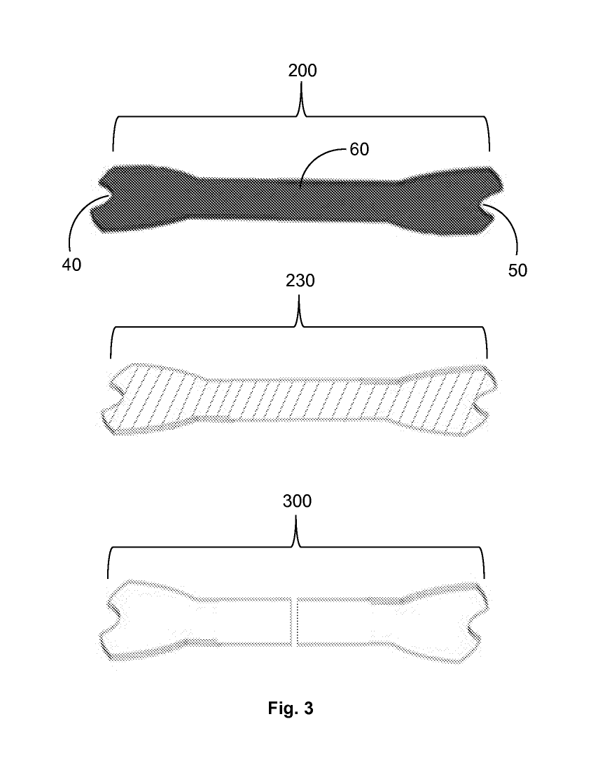

Turning to FIG. 3, the truss member 10 includes a flexible strip of base layer material 200 with an upper surface and lower surface defining the first end region 40 and second end region 50 and the intermediate segment 60. The base layer material 200 may include an elastic element that is adjustable to an increased length of the entire truss member 10.

In the preferred embodiment, the engagement means is a flexible adhesive material with memory 230 along the lower surface of the flexible base layer material 200 such that the nasal dilator can be removably affixed to the cheek of the user by pulling the area surrounding the alar facial groove 100 in both an outward and upward direction towards the anterior tragus of the ear 110 when the elastic element is reduced in length from the increased length.

In the preferred embodiment, the flexible adhesive material with memory is an elastic material often known as “Kinesiology” tape material or elastic therapeutic tape. It is generally an elastic cotton strip with an acrylic adhesive that can stretch about 140% of its original length. As a result, when applied to the cheek and placed by the tragus of the ear when stretched out, it will “recoil” after being applied and therefore create a pulling force on the skin that it is being applied to—in this instance opening the nasal passages. Other types of materials may accomplish the same goal, including but not limited to a woven fabric with memory covering the adhesive layer.

In the preferred embodiment, the base layer 200 has a periphery at least equal to the periphery of the adhesive layer 230, with at least a portion of the base layer 200 periphery position inside the periphery of the truss 10.

The invention may further comprise a protective release liner material 300 wherein the adhesive material 230 is disposed between the protective release liner 300 and the flexible strip of base material 200. The liner material 300 may be the same size and shape as the base material 200. The base layer material 200 may be pigmented to be anti-glare or anti-reflective.

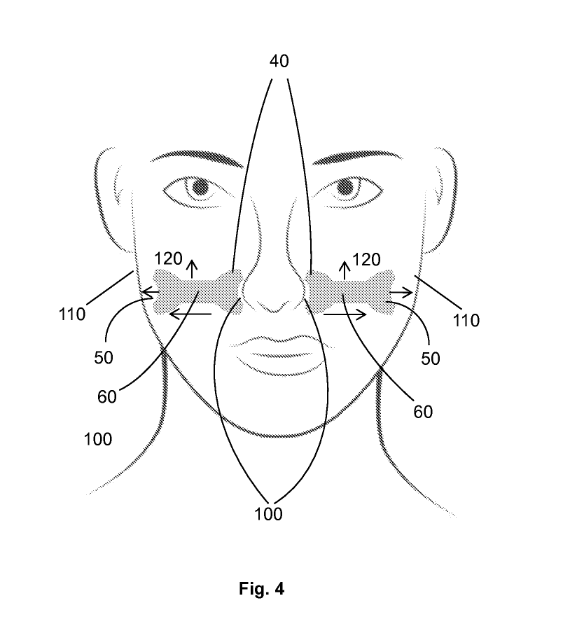

A pair of nasal dilators may be used as a system and method for dilating nasal passages as shown generally in FIG. 4 wherein a single body truss 10 having a first end region 40 that engages the alar facial groove 100; a second end region 50 that engages the area anterior to the tragus of the ear 110; and an intermediate segment 60 that connects the first end region 40 and second end region 50 wherein the intermediate segment 60 is configured to traverse the check located between the alar facial groove 100 and area anterior to the tragus of the ear 110. The truss member may include a flexible strip of base material 200 with an upper 210 and lower surface 220 defining the first end region 40 and second end region 50 and the intermediate segment 60. The engagement means in the preferred embodiment is an adhesive material 230 along the lower surface of the flexible base material 200.

The adhesive material 230 is placed at the first end region 40 along the alar facial groove 100 and at the second end region 50 along the area anterior to the tragus of the ear 110.

While the dimensions of the truss may be variable, in the preferred embodiment, the truss is from about 1.00 to about 4.00 inches in length and from about 0.25 to about 2.50 inches in height with the middle segment from about 0.25 to about 2.00 inches in length.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the method (and components of the individual operating components of the method) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections might be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

What is claimed is:

1. An external nasal dilator formed as a single body truss member, the truss member comprising:
a. An upper and lower surface;
b. A first end region with an engagement means to engage the alar facial groove and a second end region with an engagement means to engage the area anterior to the tragus of the ear; and
c. An intermediate segment connecting the first and second regions and configured to traverse the cheek located between the alar facial groove and area anterior to the tragus of the ear.

2. The external nasal dilator of claim 1 wherein the truss member includes a flexible strip of base layer material with an upper and lower surface defining the first and second end regions and the intermediate segment.

3. The external nasal dilator of claim 2 wherein the base layer material includes an elastic element that is adjustable to an increased length.

4. The external nasal dilator of claim 3 wherein the engagement means is a flexible adhesive material with memory along the lower surface of the flexible base layer material such that the nasal dilator can be removably affixed to the cheek of the user.

5. The external nasal dilator of claim 4 wherein the area surrounding the alar facial groove is pulled by the dilator in both an outward and upward direction towards the anterior tragus of the ear when the elastic element is reduced in length from said increased length.

6. The external nasal dilator of claim 3 wherein the base layer has a periphery at least equal to the periphery of the adhesive layer, at least a portion of said periphery position inside the periphery of the truss.

7. The external nasal dilator of claim 6 further comprising a protective release liner material such that the adhesive material is disposed between the protective release liner and the flexible strip of base material.

8. The external nasal dilator of claim 7 wherein the protective release liner material is the same size and shape as the base material.

9. The external nasal dilator of claim 2 wherein the base layer material is pigmented to be anti-glare.

10. The external nasal dilator of claims 2 wherein the base layer material is pigmented to be anti-reflective.

11. A system for dilating nasal passages, the system comprising:
a. A pair of external nasal dilators, each dilator formed as a single body truss member, the truss member comprising:
i. An upper and lower surface;
ii. A first end region with an engagement means to engage the alar facial groove and a second end region with an engagement means to engage the area anterior to the tragus of the ear; and
iii. An intermediate segment connecting the first and second regions and configured to traverse the cheek located between the alar facial groove and area anterior to the tragus of the ear.

12. The system of claim 11 wherein each truss member includes a flexible strip of base layer material with an upper and lower surface defining the first and second end regions and the intermediate segment.

13. The system of claim 12 wherein the base layer material or each truss includes an elastic element that is adjustable to an increased length.

14. The system of claim 13 wherein the engagement means is a flexible adhesive material with memory along the lower surface of the flexible base layer material such that each nasal dilator can be removably affixed to the opposing cheeks of the user.

15. The system of claim 14 wherein the opposing areas surrounding the alar facial groove is pulled by each dilator in both an outward and upward direction towards the opposing areas of the anterior tragus of the ear when the elastic element is reduced in length from said increased length.

16. The system of claim 12 wherein the base layer has a periphery at least equal to the periphery of the adhesive layer, at least a portion of said periphery position inside the periphery of the truss.

17. The system of claim 12 further comprising a protective release liner material such that the adhesive material is disposed between the protective release liner and the flexible strip of base material.

18. The system of claim 13 wherein the base layer material is pigmented to be anti-glare.

19. The system of claim 13 wherein the base layer material is pigmented to be anti-reflective.

20. A method of dilating nasal passages, the method comprising:
a. Providing a first truss member having (1) a first end region with an engagement means to engage the alar facial groove, (2) a second end region with an engagement means to engage the area anterior to the tragus of the ear and (3) an intermediate segment connecting the first and second regions wherein the intermediate segment is configured to traverse the check located between the alar facial groove and area anterior to the tragus of the ear and wherein the truss member includes a flexible strip of base material with an upper and lower surface defining the first and second end regions and the intermediate segment and wherein the engagement means is a flexible adhesive material with memory along the lower surface of the flexible base material;
b. Placing the adhesive material at the first end region along the alar facial groove; and
c. Placing the adhesive material at the second end region along the area anterior to the tragus of the ear.
